(19) United States
(12) Patent Application Publication
(10) Pub. No.: US 2005/0089453 A1
(43) Pub. Date: Apr. 28, 2005

(54) Flame-free aromatizer burner

(76) Inventor: Shu-Li Huang, Taipei (TW)

Correspondence Address: Sathelyne Ltd., P.O. Box No. 6-57, Junghe, Taipei 235, TW

(21) Appl. No.: 10/691,492

(22) Filed: Oct. 24, 2003

(51) Int. Cl. A61L 9/03

(52) U.S. Cl. 422/125

(57) ABSTRACT

A flame-free aromatizer burner is fastened at an aromatherapy essence ceramic core using the ceramic core fixing assembly; the electrothermal device is joined with a control circuit board; and the electrothermal device and the control circuit board heat up the ceramic core to 1100 degrees Centigrade. Essence particles in the aromatherapy essence ceramic core are aerosolized by a high temperature to produce an aroma, and a power supply is automatically cut off when having completed a heating process. The structure is capable of aerosolizing essence particles without requiring a flame, thereby eliminating potential explosions caused by igniting the ceramic core using a flame.

### BACKGROUND OF THE INVENTION

(a) Field of the Invention

The invention relates to a flame-free aromatizer burner, and more particularly, to a structure capable of aerosolizing essence particles without requiring flames, thereby eliminating potential explosions caused by igniting an aromatherapy essence ceramic core using a flame.

(b) Description of the Prior Art

A so-called aromatherapy essence is a volatile and highly concentrated fragrant object. Using various chemical or physical means, an aromatherapy essence is extracted from roots, stems, leaves, seeds or blossoms of plants, and is commonly referred to as a unilateral essence. Due to immense work stress nowadays, aromatherapy essences are frequently used by modern people in order to clear one's mind for focusing concentration, or to relax and relieve oneself from anxiety for further putting one to better sleep.

It is known that an aromatherapy essence is insoluble in water substances but soluble in alcoholic substances. Therefore, an isopropyl alcohol is generally selected as a solvent thereof. Referring to FIG. 1, a mixture solute c from an aromatherapy essence and an isopropyl alcohol is placed in an aromatherapy essence bottle b. The aromatherapy essence bottle b is arranged with a lamp core a at an interior thereof, wherein a portion of the core a is dipped in the mixture solute c. The aromatherapy essence bottle b also has an aromatherapy essence ceramic core d at a bottle opening thereof. The aromatherapy essence ceramic core d is joined with one end of the lamp core a. Using capillarity of the lamp core a, the mixture solute b in the aromatherapy essence bottle b is ascended to reach the aromatherapy essence ceramic core d. Because the aromatherapy essence ceramic core d is made of a porous ceramic material formed by particles having spaces in between, the aromatherapy essence ceramic core d is provided with good permeability and absorbability, and spaces within the aromatherapy essence ceramic core d become filled with the mixture solute c. A flame is utilized to ignite the aromatherapy essence ceramic core d at the aromatherapy essence bottle b, so as to burn the isopropyl alcohol contained in the mixture solute c. The flame is put out when the aromatherapy essence ceramic core d is heated up by the flame, and the aromatherapy essence is vaporized into air through the aromatherapy essence ceramic core d having a high temperature.

However, an interior of the aromatherapy ceramic core d lacks sufficient room for burning, and accidental explosions are often resulted from pressure unbalance regarding in and out of the aromatherapy essence bottle b. In addition, particles of the aromatherapy essence are inflammable but are only diffused into air via combustion of the isopropyl alcohol vapors. Hence, aromatherapeutic effects can be relatively affected in the absence of sufficient room for producing vapor needed by combustion.

### SUMMARY OF THE INVENTION

In the view of the aforesaid shortcomings of the prior aromatherapy essence ceramic core ignited using a flame, the primary object of the invention is to provide a structure capable of aerosolizing essence particles without using any flames, thereby eliminating potential explosion hazards caused by igniting an aromatherapy essence ceramic core with a flame.

To accomplish the aforesaid object, the flame-free aromatizer burner according to the invention comprises an upper casing, a lower casing, a ceramic core fixing assembly and an electrothermal device. The ceramic core fixing assembly and the electrothermal device are located in a housing formed by the upper casing and the lower casing. The invention is characterized that, the electrothermal device of the flame-free aromatizer burner is connected with a control circuit board; the aromatherapy essence ceramic core fixing assembly is for fastening the flame-free aromatizer burner to an aromatherapy essence ceramic core; and the ceramic core is heated to 1100 degrees Centigrade using the electrothermal device and the control circuit. An aroma is produced by essence particles in the aromatherapy essence ceramic core heated and aerosolized, and a power supply is automatically cut off when having completed a heating process.

According to the aforesaid structure, the flame-free aromatizer burner according to the invention is capable of aerosolizing essence particles without requiring a flame, thereby eliminating potential explosions caused by igniting an aromatherapy essence ceramic core with a flame.

### BRIEF DESCRIPTION OF THE DRAWINGS

FIG. 1 shows an example of a prior aromatherapy essence ceramic core.

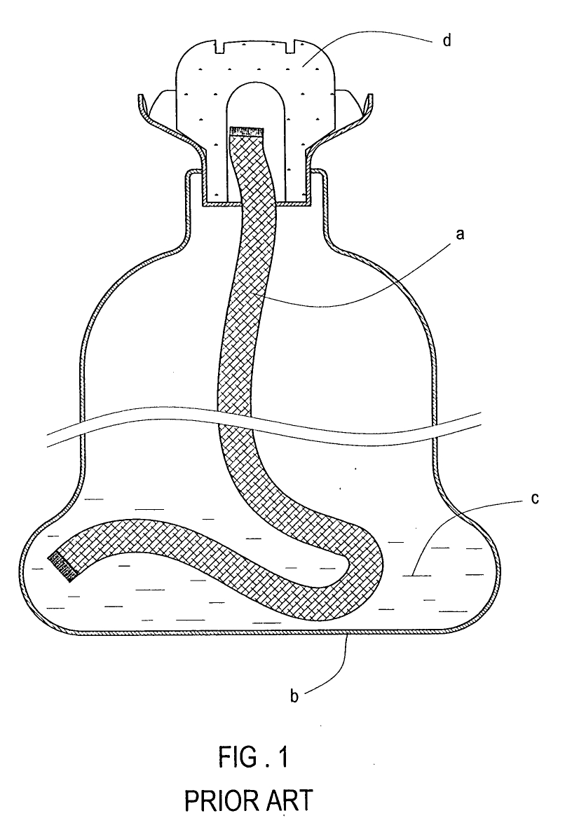

FIG. 2 shows an elevational schematic view according to the invention.

FIG. 3 shows a circuit diagram of an internal control circuit board according to the invention.

FIG. 4 shows an embodiment according to the invention.

### DETAILED DESCRIPTION OF THE PREFERRED EMBODIMENTS

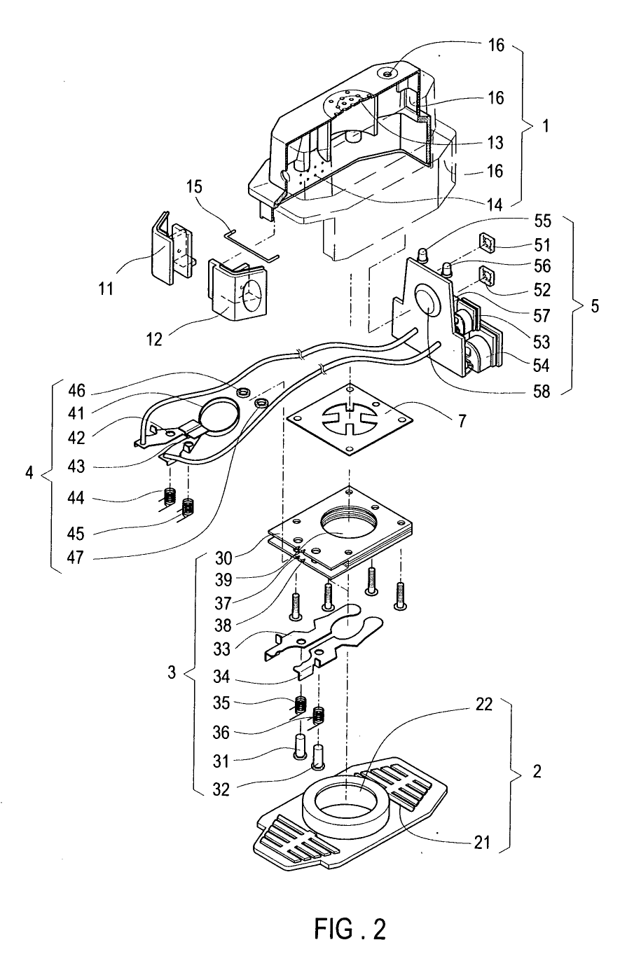

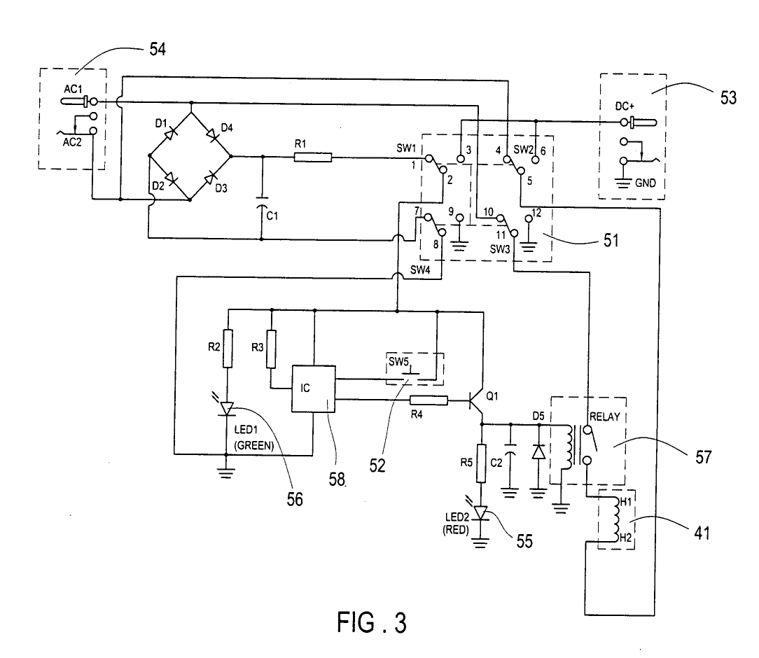

To better understand the structure, devices and characteristics of the invention, descriptions of a preferred embodiment shall be given with the accompanying drawings below. Referring to FIGS. 2 and 3, a flame-free aromatizer burner according to the invention comprises an upper casing 1, a lower casing 2, a ceramic core fixing assembly 3 and an electrothermal device 4. The ceramic core fixing assembly 3 and the electrothermal device 4 are located in a housing formed by the upper casing 1 and the lower casing 2. The upper casing has a plurality of orifices 13 and 14 at upper and lateral surfaces thereof, respectively. The lower casing 2 has a round opening 22 at a center thereof for corresponding with an aromatherapy essence ceramic core, and is provided with a plurality of ventilation railings 21 at two sides thereof. Characteristics of the invention shall be illustrated as below.

The ceramic core fixing assembly 3 is a spring clamping structure; and is consisted of a locating board 30, a left clamping element 33, a right clamping element, a left locating post 31, a right locating post 32, a left spring 35 and a right spring 36. Each of the left clamping element 33 and the right clamping element 34 has one end thereof formed as a crescent aperture, and the crescent apertures are corresponded with a central opening 39 of the locating board 30. For assembly, the left locating post 31 and the right locating post 32 are penetrated through the left spring 35 and the right spring 36 in an upward direction, respectively; and are next penetrated through holes of the left clamping element 33 and the right clamping element 34, respectively. The left clamping element 33 and the right clamping element 34 are arranged with the crescent apertures thereof facing each other. The left locating post 31 and the right locating post 32 are penetrated through lower holes of the locating board 30. Each of the left spring 35 and the right spring 36 is hooked at notches 37 and 38, respectively, while the other ends thereof are butted against vertical end portions of the left clamping element 33 and the right clamping element 34, respectively.

The electrothermal device 4 of the flame-free aromatizer burner is an elastic dilating ring structure. The electrothermal device 4 is consisted of a ring-shaped silicon thermal conductive body 41, a left rocking arm 42, a right rocking arm 43, a left spring 44 and a right spring 45. The left rocking arm 42 and the right rocking arm 43 are made of electric conductive metal materials. For assembly, the left rocking arm 42 and the right rocking arm 43 are inserted through and positioned at a middle lining layer of the locating board 30. The left locating post 31 and the right locating post 32 are penetrated through the left spring 44 and the right spring 45 in an upward direction, respectively; and are next penetrated through the left rocking arm 42 and the right rocking arm 43, and the washers 46 and 47, respectively. The left locating post 31 and the right locating post 32 are fastened at an upper layer of holes of the locating board 30. One end of each of the left spring 44 and the right spring 45 are hooked at the notches 37 and 38, respectively, while the other ends thereof are butted at vertical end portions of the left rocking arm 42 and the right rocking arm 43, respectively. A stopping plate 7 is fixed at an upper surface of the locating board 30. A front end of the left rocking arm 42 is joined with one end of the ring-shaped silicon thermal conductive body 41, and a front end of the right rocking arm 43 is joined with the other end of the ring-shaped silicon thermal conductive body 41, such that the ring-shaped silicon thermal conductive body 41 is surrounded at an outer periphery of the central opening 39. Rear ends of the left rocking arm 42 and the right rocking arm 43 are joined with two wires extended from the control circuit board 5. The control circuit board 5 has electric components including a power selection switch 51, an activation switch 52, a direct-current power receptacle 52, an alternating-current power receptacle 54, a red light emitting diode 55, a green light emitting diode 56, a relay 57 and an integrated circuit 58.

When the aforesaid ceramic core fixing assembly 3, the electrothermal device 4 and the stopping plate 7 are assembled, using screws in conjunction with holes at edges of the locating board 30 and the stopping plate 7, the locating board 30, the stopping plate 7, the ceramic core fixing assembly 3 and the electrothermal device 4 are fastened at locating screw openings in the upper casing 1. The control circuit board 5 is placed at one side in the upper casing 1, and the power selection switch 51, the activation switch 52, the direct-current power receptacle 53, the alternating-current power switch 54, the red light emitting diode 55 and the green light emitting diode 56 are arranged at individual openings 16. End portions of the left rocking arm 42, the right rocking arm 43, the left clamping element 33 and the right clamping element 34 are disposed with flexible buttons 11 and 12 that are joined with a connecting strip 15. The lower casing 2 is fastened at a lower portion of the upper casing 1 to complete the assembly of the structure.

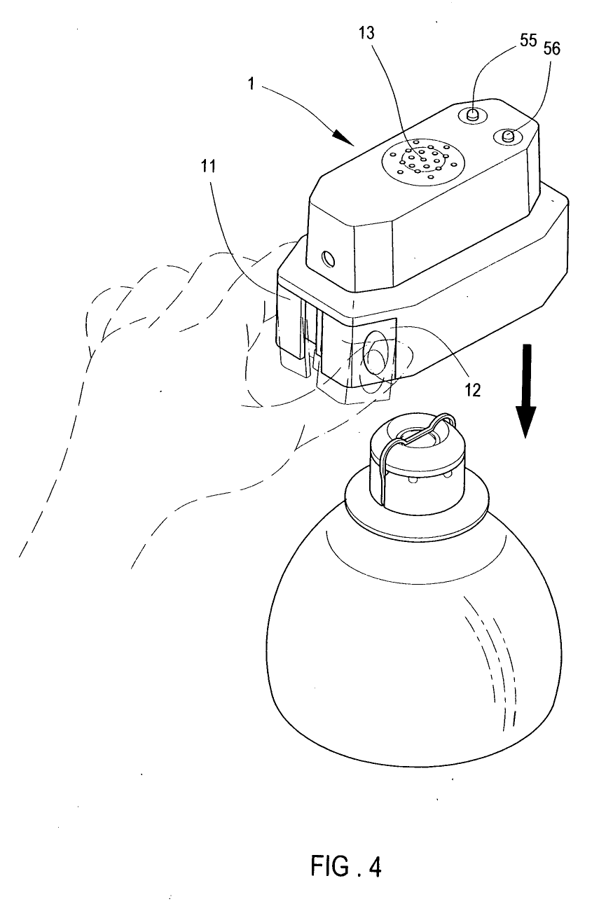

Referring to FIGS. 2, 3 and 4, to use the flame-free aromatizer burner, the flexible buttons 11 and 12 are pressed by fingers of a user, so as to have the left clamping element 33, the right clamping element 34, the left rocking arm 42 and the right rocking arm 43 to stretch outward. For that the left rocking arm 42 and the right rocking arm 43 are stretched outward, the ring-shaped structure surrounded by the ring-shaped silicon thermal conductive body 41 is also dilated. After being dilated, the ring-shaped silicon thermal conductive body 41 has a size suitable for accommodating an aromatherapy essence ceramic core. The aromatherapy essence ceramic core is put into the round opening 22 in an upward direction. The flexible buttons 11 and 12 are then released, so that the aromatherapy essence core is clamped and stabilized by the left clamping element 33 and the right clamping element 34 of the ceramic core fixing assembly 3, and is tightened and surrounded by the ring-shaped structure of the ring-shaped silicon thermal conductive body 41. Using the power selection switch 51, direct-current or alternating-current may be selected providing the flame-free aromatizer burner with a current. In case that alternating-current is to be used, a supply current is converted into direct-current by a rectification current at the control circuit board 5, and the direct-current is entered into a power input end of the integrated circuit 58. At this point, the integrated circuit 58 is at a standby mode, and outputs a voltage for illuminating the green light emitting diode 56 to notify a user that the device is ready to use. When the user presses the activation switch 52, a voltage is passed through the switch 52 to enter the integrated circuit 58, and an input voltage is detected by the integrated circuit 58. An internal circuit starts timing, inputs a voltage into a control end of the relay 57 for conducting a contact end thereof, and illuminates the red light emitting diode 55. Thus, a current loop is formed by the ring-shaped silicon thermal conductive body 41 and the input current supply. The current heats the ring-shaped silicon thermal conductive body 41 from a room temperature to 1100 degrees Centigrade within four seconds, and the aromatherapy essence core becomes heated. After a certain period timed by the integrated circuit 58, the voltage inputted into the control end of the relay 57 is automatically cut off. The ring-shaped silicon thermal conductive body 41 no longer receives a current supply, and ceases heating the aromatherapy essence ceramic core as well as stop illuminating the red light emitting diode 55 and the green light emitting diode 56, so as to indicate that a heating process is completed. At his point, essence particles in the aromatherapy essence ceramic core are heated and aerosolized, and the user may then remove the power supply from the direct-current power receptacle 53 or the alternating-current power receptacle 54.

According to the aforesaid flame-free aromatizer burner, electricity is utilized for the ring-shaped silicon thermal conductive body 41 to produce a high temperature for further heating the aromatherapy essence ceramic core. The ring-shaped silicon thermal conductive body 41 heats from a room temperature to 1100 degrees Centigrade within four seconds. Owing to a rapid raise in temperature, neither flames nor or smoke is produced during the heating process. In addition, being surrounded at a periphery of the aromatherapy essence ceramic core, the ring-shaped silicon thermal conductive body 41 not only avoids hazards brought about by combustion but also evenly distributes heat at the aromatherapy ceramic core for obtaining enhanced heating effects. Using a high temperature, the ring-shaped silicon thermal conductive body 41 promotes complete aerosolization of the essence particles in the aromatherapy essence ceramic core, thereby effectively preventing small pores in the aromatherapy essence ceramic core from being blocked.

Conclusive from the above, the flame-free aromatizer burner is capable of preventing potential hazards caused by ignition as well as preventing the pores in the aromatherapy essence ceramic core from being blocked. It is of course to be understood that the embodiment described herein is merely illustrative of the principles of the invention and that a wide variety of modifications thereto may be effected by persons skilled in the art without departing from the spirit and scope of the invention as set forth in the following claims.

1. A flame-free aromatizer burner comprising an upper casing, a lower casing, a ceramic core fixing assembly and an electrothermal device;
wherein the ceramic core fixing assembly and the electrothermal device are located in a housing formed by the upper housing and the lower housing; and
the characteristics being that, the flame-free aromatizer burner is fastened at an aromatherapy essence ceramic core using the ceramic core fixing assembly in a spring clamping structure; the electrothermal device is an elastic dilating ring structure, and has a dilating ring made of a ring-shaped silicon thermal conductive material; the electrothermal device is joined with a control circuit board; using the aforesaid structure, the flame-free aromatizer burner is fastened at the ceramic core using the ceramic core fixing assembly and is surrounded by the ring-shaped silicon thermal conductive body; and a current is conducted through the ring-shaped silicon thermal conductive body using the control circuit board, thereby producing a high temperature for heating the aromatherapy essence ceramic core.

2. The flame-free aromatizer burner in accordance with claim 1, wherein the control circuit board having an integrated circuit for controlling time and a relay, thereby controlling a heating time of the ring-shaped silicon thermal conductive body relative to the aromatherapy essence ceramic core.

3. The flame-free aromatizer burner in accordance with claim 1, wherein the control circuit board further having a light emitting diode for indicating usage status.
